(19) United States
(12) Patent Application Publication
(10) Pub. No.: US 2007/0100363 A1
(43) Pub. Date: May 3, 2007

(54) Aortic lancet

(76) Inventors: Michael L. Dollar, Garland, TX (US); Victor A. Dubuclet IV, Dallas, TX (US); David T. Ernst, Allen, TX (US)

Correspondence Address: CARSTENS & CAHOON, LLP, P O BOX 802334, DALLAS, TX, 75380, US

(21) Appl. No.: 11/260,639

(22) Filed: Oct. 27, 2005

(51) Int. Cl. A61B 17/32

(52) U.S. Cl. 606/181

(57) ABSTRACT

A system for making a precise incision and circular hole in a vessel wall, such as the aorta, that eliminates lateral side notches from the aortotomy. In one aspect, the system includes a surgical knife or lancet having a blade surrounded by a retractable shield. In one aspect, the system includes a tissue punch having a rotating circumferential edge for receiving a parabolic-shaped anvil having an anvil cutting edge. The anvil is placed through an incision made by a knife and actuated to produce a hole in the vessel wall.

### BACKGROUND OF THE INVENTION

1. Technical Field

The present invention is directed generally to methods and apparatus for effectuating surgical incisions. More specifically, the present invention is related to methods and apparatus for creating a hole in a vessel wall including an aorta wall.

2. Description of Related Art

During surgical procedures, such as placement of a ventricular assist device, blood vessel anastomosis, aortotomy, gastrotonomy, enterotomy, or access to other hollow organs and vessels, it is useful to have specialized tools to create a circular opening in the wall of the vessel or organ. Tools have been developed for use in surgery that create such openings. For example, often the goal of heart surgery is to produce a blood flow path around the diseased areas of coronary arteries. A saphenous vein graft can be used by forming an opening in the wall of an ascending aorta, and anastomosing the opening to a proximal end of the saphenous vein. To form the opening in the wall of the aorta, an incision can be made using surgical scalpels and/or scissors. An aortic punch can then be used in order to attempt to obtain a clean, accurate somewhat larger opening in the aortic wall. Obtaining a clean and accurate opening is extremely important since an opening which is not formed cleanly and accurately often is frayed, albeit microscopically. As a result, the connection of the proximal end of the saphenous vein thereto may not be as reliable, and complications during or after surgery may result. Because heart surgery necessarily often entails a difference between life and death of a patient, it is extremely important to maximize the probability of success of every aspect of the surgical procedure. To this end, it is desirable to try to obtain as clean and as accurate an opening as possible in the wall of the aorta before grafting the saphenous vein thereto.

Once such procedure is described and illustrated in U.S. Pat. No. 6,187,022. A conventional linear incision along an aorta vessel utilizing a conventional scalpel is first made. Such procedure requires the surgeon to make an educated guess as to the length of the incision. After the incision is made, a punch is inserted into the vessel wall through the incision to make a clean circular hole in the vessel wall. Unfortunately, the diameter of the hole made in the vessel wall is sometimes less than the length of the educated-guess incision made by the scalpel resulting in lateral nicks protruding from opposite sides of the hole in the vessel wall, as illustrated by FIG. 2 of the '022 Patent. These nicks necessitate special suturing to prevent blood leakage, and add points of weakness at the site of the vessel graft. Alternatively, a surgeon may make an incision that is smaller than the diameter of the punch such that lateral nicks might be avoided. However, such an incision necessitates stretching of the tissue for insertion of the aortic punch anvil. The stretching of the tissue can decrease the patency of the resulting graft or cause aortic dissection wherein the layers of the aortic wall separate from one another. The '022 Patent's solution to this problem is to use a cruciate-shaped blade to make a cross-shaped incision for the initial incision into the vessel wall, and subsequently completing the opening with an aortic punch.

An additional problem a surgeon faces with making such incision is that the heart, in some procedures, is not arrested and is permitted to beat, which can impart some movement on the vessel that the surgeon is attempting to make an incision. Such movement can make an accurate incision more difficult. Further, if a surgeon applies too much pressure and pushes the scalpel or blade too far into the vessel, damage to the opposite or back wall of the vessel can occur. Consequently, a need exists for an improved incision device or lancet. The improved device should protect the surgeon's fingers from being cut with the blade, and it should provide a way to minimize or eliminate cuts that could be accidentally made during surgery. The improved device should minimize or eliminate nicks outside the diameter of the punched hole in the vessel wall and should provide a clean cut.

As discussed above, after an incision is made, an aortic punch is used to obtain the opening in the aortic wall. One example of such punch is illustrated by U.S. Pat. No. 5,827,316 (the '316 Patent). One problem with prior art punches is that the distal end of the anvil is typically flat, or has a tip with a vertex having a large angle. As a result, sufficient pressure must be supplied to penetrate the incision. As the amount of pressure required increases, the potential for an undesirable fissure or tear in the vessel wall increases as well. Further, additional pressure can cause undesirable stretching of the tissue remaining after the hole in the vessel has been made. Consequently, a need exists for an aortic punch that can minimize or eliminate fissures, or tears, and/or stretching that can occur in a vessel wall.

### SUMMARY OF THE INVENTION

The present invention relates to an improved system for making a precise incision and circular hole in a vessel wall. In one aspect, the system comprises a lancet having a retractable shield surrounding a blade. The retractable shield helps to prevent any accidental cutting that can occur during surgical conditions. When an incision is made, the shield simultaneously retracts as the blade penetrates the front vessel wall. The blade length is less than the inside diameter of the vessel wall to prevent accidental penetration of the back vessel wall. In one aspect, the system comprises a punch having a tapered cutter with a circumferential edge for receiving an anvil cutting edge. The anvil is placed through the incision in the front vessel wall and actuated to cause a helical shearing force as the anvil cutting edge axially slides past the rotary circumferential cutting edge to produce a clean-cut hole in the vessel wall.

The above as well as additional features and advantages of the present invention will become apparent in the following written detailed description.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is an exploded perspective view of the lancet in accordance with one embodiment of the present invention.

FIG. 2 is a perspective view of the lancet in accordance with one embodiment of the present invention.

FIG. 3 is a partial side view of the lancet inserted in a vessel wall in accordance with one embodiment of the present invention.

FIG. 4 is a partial side view of the lancet in accordance with one embodiment of the present invention.

FIG. 5 is a side view of the aortic punch in accordance with one embodiment of the present invention.

FIG. 6 is a partial side view of the aortic punch inserted in a vessel wall in accordance with one embodiment of the present invention.

### DETAILED DESCRIPTION

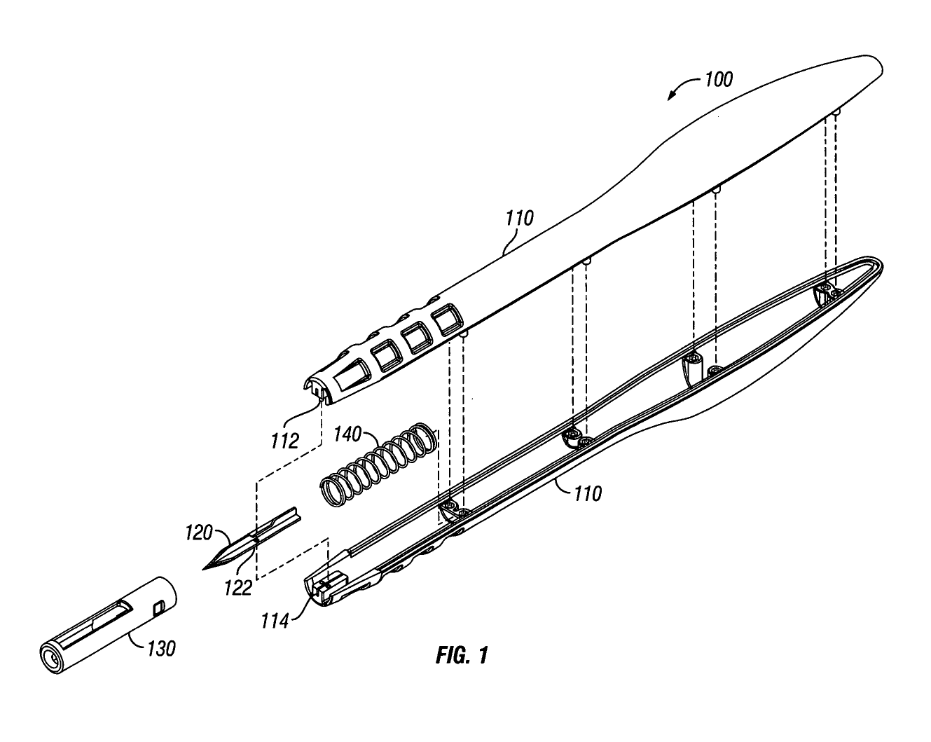

FIG. 1 is an exploded perspective view of the lancet in accordance with one embodiment of the present invention. The lancet 100 comprises a blade 120 attached to a handle 110. In one embodiment, the handle comprises a first section and a second section that can be removably snap-fit together. In one embodiment, the first section of the handle 110 comprises at least one and preferably two male members 112 disposed through an aperture 122 in the blade 120 and into a female member 114 in the second section to attach the blade 120 to the handle 110. The blade 120 thereby protrudes from the handle 110. A retractable blade shield 130 is slidably attached to the handle 110 and surrounds the blade 120. In one embodiment, the blade shield 130 is clear or transparent so that the blade 120 is visible.

The blade shield 130 helps to minimize accidental cutting that can occur in surgical conditions. For example, it should be noted that during surgical conditions, slippery fluids such as blood and sudden movements created by a beating heart in conjunction with the decreased tactile function that can occur with a surgeon wearing latex gloves create conditions that can result in an accidental cutting. Further, the blade shield 130 can be gripped by a surgeon's fingers to aid in initial placement of the blade 120. Thus, the blade shield 130 can help protect the surgeon's fingers during surgical conditions and/or ensure that an incision is made only at the desired place on the vessel. In one embodiment, the retractable blade shield 130 is kept in an extended position around the blade 120 by a spring, or other equivalent resistance means. In one embodiment, the spring 140 comprises a resistance that is less than the insertion force required to make an incision into a vessel.

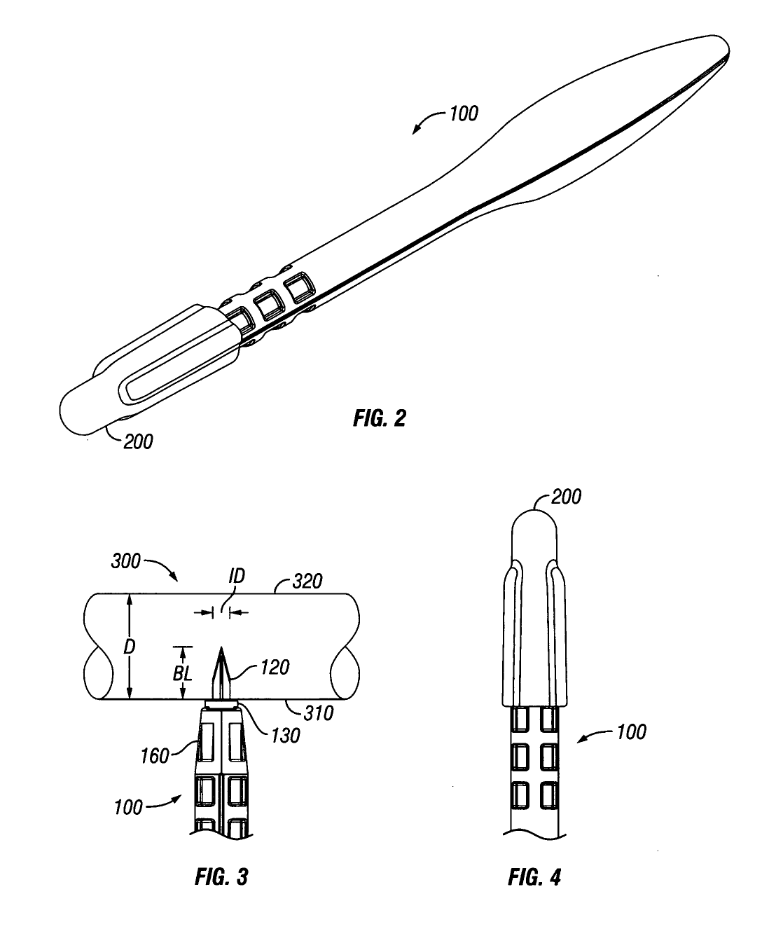

FIG. 2 is a perspective view of the lancet in accordance with one embodiment of the present invention. FIG. 4 is a partial side view of the lancet in accordance with one embodiment of the present invention. Referring to FIGS. 2 and 4, a protective cover 200 can be press-fit over the retractable blade shield and blade and over a portion of the handle as an additional safety precaution. Further, the protective cover 200 can protect the blade 122 from potential contaminants when the lancet 100 is not in use.

FIG. 3 is a partial side view of the lancet inserted in a vessel wall in accordance with one embodiment of the present invention. As depicted in FIG. 3, the lancet is shown without a protective cover and with the retractable shield 130 in the fully retracted position. The blade 120 having a blade length BL is thereby exposed within the vessel 300. The blade length BL is defined as the length of the blade 130 that is exposed when the blade shield is fully retracted. In one embodiment, the blade length BL is between about 1 millimeter and about 20 millimeters and more preferably between about 5 millimeters and about 15 millimeters. In one embodiment, the blade length BL is dimensioned such that during blade penetration of the front vessel wall 310, the back wall 320 cannot be penetrated. Consequently, in one embodiment, the blade length BL is substantially less than or equal to the inner wall vessel diameter D. In one embodiment, the blade comprises a primary and secondary blade similar to that disclosed in U.S. Pat. No. 6,187,022 and makes a cruciate-shaped incision having an incision diameter ID. In one embodiment, the end of the handle is tapered 160 to provide a surgeon with a better finger grip.

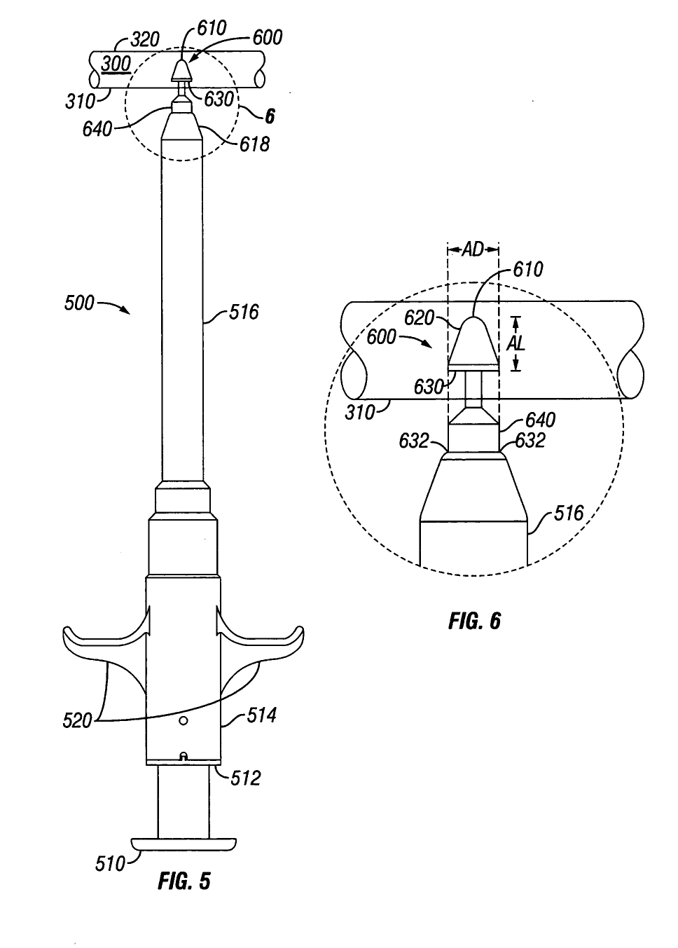

FIG. 5 is a side view of the aortic punch in a vessel wall in accordance with one embodiment of the present invention. FIG. 6 is an enlarged partial side view of the aortic punch in accordance with one embodiment of the present invention. Referring to FIG. 5 and FIG. 6, the aortic punch 500 is shaped and designed to be used by a surgeon using one of his or her hands. The aortic punch includes a thumb button 510 pushable into an opening 512 in a finger grip body 514 which is a non-rotating member. A pair of finger seats 520 can be attached to the finger grip body 514 and can be shaped to be engaged by a surgeon's fingers. To operate the aortic punch 500, a surgeon can grab the punch in his or her hand, place a thumb on the thumb button 510 and place both an index finger tip and a middle finger tip on the finger seats 520. After an incision has been made by a lancet, the surgeon can maneuver the aortic punch 500 so that the parabolic-shaped anvil 600 on the end of the shaft is inserted into the incision in the front vessel wall 310 (shown in FIG. 3).

After the anvil 600 is inserted into the incision in the front vessel wall 310, the surgeon can push on the thumb button 510 with his or her thumb while holding the finger seats 520 with his or her fingers. As the thumb button 510 is pushed into the finger grip body 514 through the opening 512, the hollow body member 516 attached to a tapered cutter 618 having a sharp circumferential edge 632 simultaneously rotates as the piston 640 and anvil 600 moves downward through the opening defined by the sharp circumferential edge 632. An opening in the front vessel wall 310 is created by a helical shearing force as the anvil cutting edge 630 axially slides past the rotating circumferential cutting edge 632. The helical shearing force imparted creates a clean-cut hole in the vessel wall. The helical shearing force also minimizes or eliminates fissures, tears, and/or stretching in the vessel area adjacent the hole. The diameter of the tapered piston 640 can be sized so as to prevent fluids and vessel tissue from entering the hollow body member 516 while permitting the piston 640 to slidably move in the axial direction within the tapered cutter 618.

The parabolic shaped anvil 600 comprises a vertex 610 at the distal end of the anvil and an anvil cutting edge 630 having an anvil diameter AD at the proximal end of the anvil 600. One advantage of the parabolic shape is that it permits easy perpendicular insertion of the anvil 600 through the incision which can help to minimize or eliminate stretching in the remaining tissue adjacent the hole formed by the anvil. The slope of the anvil sides 620 determines the penetration angle at which the anvil enters the vessel wall 310. A smaller penetration angle (e.g. narrower parabola) results in less insertion force of the anvil 600 into the vessel which minimizes undesirable vessel fissures and is less likely to cause undesirable tearing upon insertion. Consequently, a relatively longer anvil length AL can be used to provide an anvil having the proper anvil diameter AD at the anvil cutting edge 630 and an optimal penetration angle.

In one embodiment, and referring to FIG. 3 and FIG. 6, the incision diameter ID created by the blade 120 is less than or equal to the anvil diameter AD. This advantageously provides a hole having no lateral nicks that are common place in prior art devices. Hence, the punch of the present invention can be applied to a straight or cruciate incision. One advantage of the helical shearing force provided by the parabolic-shaped anvil 600 of the present invention is that it permits an incision diameter ID to be made that is smaller than the anvil diameter AD. Smaller incision diameters are desirable because it minimizes the undesirable fissures and/or tearing in the vessel wall because less insertion force is required to make smaller incisions. The present invention thereby provides a system for making a precise incision and circular hole in the aorta that eliminates lateral side notches from the aortotomy.

While this invention has been particularly shown and described with reference to a preferred embodiment, it will be understood by those skilled in the art that various changes in form and detail may be made therein without departing from the spirit and scope of the invention.

What is claimed is:

1. A lancet comprising:
a blade having a blade length attached to a handle;
a retractable shield attached to said handle wherein said retractable shield surrounds said blade.

2. The lancet of claim 1 wherein said blade comprises a multi-bladed configuration.

3. A lancet of claim 1 wherein said blade length is dimensioned such that a back wall of a vessel opposing blade insertion cannot be penetrated.

4. The lancet of claim 1 wherein said retractable shield comprises a resistance that is less than the insertion force required to force said blade into a vessel.

5. The lancet of claim 1 wherein said retractable shield is transparent.

6. The lancet of claim 1 wherein said retractable shield further comprises a protective cover.

7. The lancet of claim 1 wherein said retractable shield uses a spring.

8. An aortic punch comprising:
a non-rotating finger grip body having a first end and a second end;
a thumb button disposed on said first end wherein said thumb button can be pressed into said non-rotating finger grip body;
a rotating hollow body member attached to said second end, wherein said hollow body member further comprises a tapered cutter having a sharp circumferential edge;
a piston disposed within said circumferential edge; and
a parabolic-shaped anvil comprising an anvil cutting edge having an anvil diameter, wherein said parabolic-shaped anvil is in communication with said piston.

9. The aortic punch of claim 8 wherein upon actuation of said thumb button, said circumferential edge rotates and said anvil cutting edge axially slides past said circumferential cutting edge.

10. A method for removing a plug of tissue from a vessel wall, said method comprising steps of:
a) piercing the wall with a lancet of claim 1 to make an incision in a front vessel wall;
b) inserting an aortic punch into said incision; and
c) actuating said aortic punch to create a hole in said wall.

11. The method of claim 10 wherein said aortic punch at step b) is the aortic punch of claim 8.

12. The method of claim 10 wherein said lancet comprises four blades and wherein said piercing at step a) creates a cruciate-shaped incision having an incision diameter.

13. The method of claim 12 wherein said punch comprises an anvil diameter and wherein said anvil diameter is greater than said incision diameter.
